(19) United States
(12) Patent Application Publication
(10) Pub. No.: US 2012/0021075 A1
(43) Pub. Date: Jan. 26, 2012

(54) DUAL-CHAMBER PACKAGING SYSTEMS FOR CANNABIS-INFUSED PRODUCTS SYSTEMS

(76) Inventors: Ida Umanskaya, Cliffside Park, NJ (US); Karina Umanskaya, Cliffside Park, NJ (US)

(21) Appl. No.: 13/189,500

(22) Filed: Jul. 23, 2011

Related U.S. Application Data
(60) Provisional application No. 61/367,799, filed on Jul. 26, 2010.

(51) Int. Cl. A61K 36/00; A61B 19/02; A61P 29/00

(52) U.S. Cl. 424/725; 206/438

(57) ABSTRACT

A dual-chamber packaging for medical marijuana-infused products and edibles providing individuals who are suffering from various health problems and diseases with an effective method for ingesting and/or topically applying medical marijuana. The invention may comprise a dual-compartmental containment device made from high-density polyethylene. The two chambers may be separate but are encased in one outer container. Professionally manufactured goods, such as lotions or oils with THC and CBD enhancers and carriers and other beneficial properties, can be kept in the first compartment, while cannabinoid fluid may be contained in the second compartment. For use in transdermal delivery of cannabinoids, an individual may first dispense the professionally-developed material and rub it onto his/her skin. Dispensaries may customize the materials used in this device to satisfy the needs dictated by their patients' specific conditions or diseases. The device provides for the possibility of transporting partially professionally produced products not only within the state, but also to the states where medical marijuana is legal (while they are not filled with cannabinoids.)

### CROSS-REFERENCE TO RELATED APPLICATION

The present application is related to and claims priority from prior provisional application Ser. No. 61/367,799, filed Jul. 26, 2010 which application is incorporated herein by reference.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

1. Field of the Invention

The present invention relates generally to the field of packaging and more specifically relates to dual-chamber packaging for cannabis-infused products.

2. Description of the Related Art

Cannabinoids are a class of chemical compounds which have been found to be useful in pain treatment. Cannabinoid receptors (CB₁ and CB₂) may be found in the human brain (CB₁) and in the immune system (CB₂). Presently, cannabinoids can be administered by smoking, vaporizing, oral ingestion, transdermal application, intravenous injection, sublingual absorption, or rectal suppository in an effort to suppress pain in patients and for other medical benefits.

Currently, there are only fourteen states in the United States that permit legal use of medical marijuana to alleviate symptoms of various health issues. Only state licensed dispensaries, alternative treatment centers and growing facilities are presently permitted to produce medical marijuana products and distribute them to the patients in need. Due to these restrictions, professional manufacturers are not permitted to produce and distribute products containing medicinal marijuana. This limits individuals who rely on medical marijuana for relief to very few methods for ingesting the marijuana, like smoking and inhaling and also having scientifically formulated and FDA registered and professionally produced products for medical use. These traditional methods, such as smoking marijuana, can cause further health complications, or may be not tolerated by the patients registered in the program.

Alternatively, many consumers may choose transdermal or oral application of cannabinoids (or combine them with food or tea and soup) to provide relief; however THC and CBD molecules are too large to penetrate skin effectively (they are larger than skin molecules will allow penetration of.) Additionally, THC and CBD are not typically water soluble, and can be destroyed by being infused in boiling water. These factors prevent users from experiencing the relieving properties of medicinal marijuana in a safe and reliable fashion. Various attempts have been made to solve the above-mentioned problems such as those found in U.S. Pat. Nos. 2008/0091139; 7,709,536; 2007/0104741; 2006/0247304; 6,132,762; 6,630,507; 7,285,687; and 2008/0064679. This prior art is representative of medical marijuana processes, storage and delivery options. None of the above inventions and patents, taken either singly or in combination, is seen to describe the invention as claimed.

Ideally, a packaging and dispensing system for cannabis-infused products should be user-friendly and safe, yet would operate reliably and be manufactured at a modest expense. Thus, a need exists for a reliable packaging and dispensing system for cannabis-infused products system such that they can be dispensed in a readily usable form to provide reliable pain relief for users and to avoid the above-mentioned problems.

### BRIEF SUMMARY OF THE INVENTION

In view of the foregoing disadvantages inherent in the known packaging and dispensing art, the present invention provides a novel concept for packaging and dispensing system for cannabis-infused products system. The general purpose of the present invention, which will be described subsequently in greater detail is to provide a reliable and safe means for purveying cannabis-infused products to provide pain relief for its users.

A packaging apparatus for medical marijuana-infused products and edibles is disclosed herein preferably comprising: a housing; a cap; a first chamber; a second chamber (optionally more chambers in alternate embodiments); and at least one nozzle (nozzle(s) able to dispense atomized liquid and/or non-atomized fluid—gel consistency or the like). First and second chambers are preferably enclosed within an inner volume of the housing. The cap and housing, when coupled, define the inner volume of the packaging apparatus. The cap, when installed, covers the nozzle(s). A two nozzle configuration is the preferred embodiment because it allows the lab which scientifically produced the formulation to seal it in one compartment so it doesn't have any insurance or other claims from patients or dispensaries. In this particular version the second compartment has to be sealed by a dispensary.

Lotions and/or oils with THC and CBD enhancers and carriers can be stored in the first chamber, and cannabinoid fluid may be contained in the second chamber. The packaging apparatus comprises high-density polyethylene (durable and cost-effective to manufacture) in preferred embodiments, however may comprise other materials in alternate embodiments. The first and second chambers may be separated by a breakable seal and the chambers are refillable with desired product(s), as mentioned previously. The first chamber and second chamber are separate (isolated) from each other; wherein the first chamber and the second chamber are suitable for sealingly-containing at least one liquid and/or solid.

In preferred embodiments the first chamber and second chamber may comprise color-coding such that they are easily differentiatable by consumers. First chamber and second chamber may also comprise labels (prescriptions, patient information or other the like.) The packaging apparatus for medical marijuana-infused products and edibles provides a means for a user to safely ingest or topically apply the medical marijuana-infused products and edibles.

Additionally, a kit is disclosed herein suitable for sale and use including the packaging apparatus having a housing; a cap; at least one nozzle; a first chamber (pre-filled or not, color-coded or not); a second chamber (pre-filled or not, color-coded or not); labels (labeling means); and a set of user instructions.

A method of using the packaging apparatus for medical marijuana-infused products and edibles to ingest or topically apply medical marijuana to provide pain relief is also described preferably comprising the steps of: dispensing, ingesting and/or topically applying the medical marijuana to provide pain relief via manipulating the nozzle(s); and refilling the first chamber; and the second chamber of the packaging apparatus when the medical marijuana-infused products have been depleted (making the system re-usable.) In this way the user is able to self-administer the medical marijuana in a safe, useable form/manner such that pain relief is thereby provided.

The present invention holds significant improvements and serves as a packaging and dispensing system for cannabis-infused products. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present invention, dual-chamber packaging system for cannabis-infused products, constructed and operative according to the teachings of the present invention.

FIG. 1 shows a perspective view illustrating a dual-chamber packaging system for cannabis-infused products in a stored/transported condition (by a user) according to an embodiment of the present invention.

FIG. 2 is a perspective view illustrating embodiments of the dual-chamber packaging system for cannabis-infused products according to the present invention of FIG. 1.

FIG. 3 is a perspective view illustrating the dual-chamber packaging system for cannabis-infused products as used to sealably-contain cannabinoid fluid separately in a chamber from dry or gel products stored in another chamber according to an embodiment of the present invention of FIG. 2.

FIG. 4 is a perspective view illustrating the dual-chamber packaging system for cannabis-infused products as used to sealably-contain cannabinoid fluids in each of the chambers according to an embodiment of the present invention of FIG. 2.

FIG. 5 is a flowchart illustrating a method of use for the dual-chamber packaging system according to an embodiment of the present invention of FIGS. 1-4.

The various embodiments of the present invention will hereinafter be described in conjunction with the appended drawings, wherein like designations denote like elements.

### DETAILED DESCRIPTION

As discussed above, embodiments of the present invention relate to a packaging and dispensing device and more particularly to a packaging and dispensing system for cannabis-infused products as used to improve the means for purveying cannabis-infused products to provide pain relief for its users. The carrier is preferably a patentable formulation made by Beilis Development laboratory, a professionally formulated and made product and even FDA registered.

The nature of the present invention is that the dual chambered device is not only for transdermal application but also for oral application. One chamber contains cannabinoid carrier that may be absorbed through the user's skin (or other suitable carrier), the other chamber may contain cannabinoid extract prepared by a dispensary or by a patient himself/herself. One chamber with a cannabinoid carrier may be a vial or when containing tea or soup it can be a double chamber tea bag, half of it contains a carrier which allows cannabinoids to not be destroyed in hot or boiled water.

For sublingual (oral) application such as with a toothpaste, the vial contains a professionally formulated and produced gel or lotion with beneficial properties and great FDA registered carriers. Thus the dispensaries in the individual states (that the product is legal within) would have professionally formulated products, not harmful but very beneficial for use, while substantially preventing homemade, unprofessionally, unsanitary formulations from being produced and dispensed. Toothpaste, when brushed against the gums promotes efficient movement of the cannabinoid molecules into the bloodstream.

Generally speaking, the dual-chamber (or multiple-chamber in alternate embodiments) packaging for medical marijuana-infused products and edibles provides an effective means for incorporating cannabinoids with previously manufactured goods, such as lotions, oils, and edible items. This innovative invention preferably features a containment mechanism including one first chamber designed to hold the aforementioned professionally formulated goods with THC and CBD cannabinoid carriers and enhancers and other necessary benefitial properties, and a second chamber intended to contain cannabinoid fluids or optionally dry medical marijuana. By pumping a nozzle at the top of the device, the two materials are simultaneously mixed and dispensed, or separately dispensed-in the case of pumping two separate nozzles-allowing transdermal or oral delivery of cannabinoids to the user.

This novel product may be ideal for consumers in need of a safe, easy method for ingesting or topically applying (gel or the like) medical marijuana to alleviate various symptoms of different diseases and health problems, and may be used only in states where use of medical marijuana is legal. The device provides a means by which cannabinoid molecules, which are normally too big to pass through the skin, are able to enter the bloodstream.

Dual-chamber (multiple-chamber) packaging for medical marijuana-infused products and edibles provides individuals who are suffering from various health problems and diseases with an effective method for ingesting or topically applying medical marijuana. The invention may comprise a dual-compartmental containment device made from high-density polyethylene. The two chambers can be separate but are preferably encased in one outer container. Professionally manufactured goods, such as lotions or oils with THC and CBD enhancers and carriers and other beneficial properties, can be kept in the first compartment, while cannabinoid fluid may be contained in the second compartment. An upper cover made to fit over the top of the device may include two pump nozzles, allowing users to access the materials in the individual chambers separately, or alternately a single nozzle may be used to dispense materials in different chambers simultaneously.

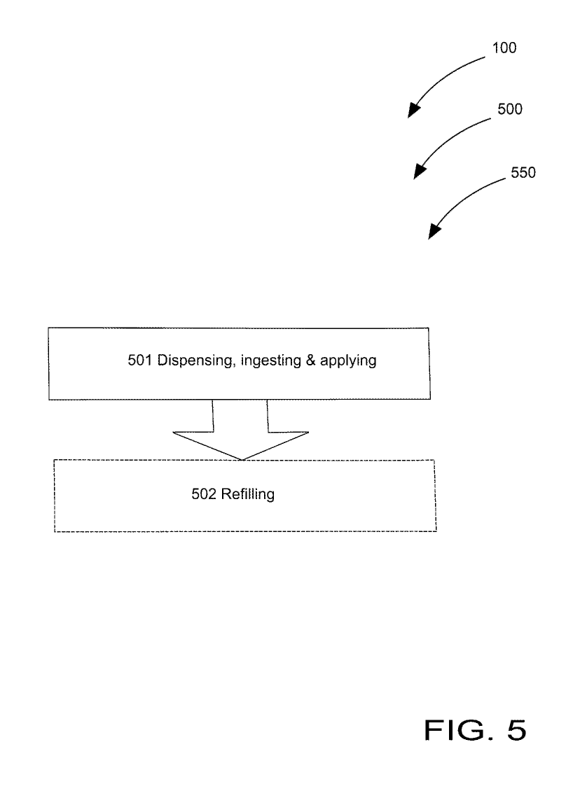

To use for transdermal delivery of cannabinoids, an individual may first dispense the professionally-developed material and rub it onto his/her skin. Then, the cannabinoid fluid can be dispensed and applied on top of the first substance. A single-nozzle design can also be available for oral-mucosal delivery of cannabinoids. Reference to a method of use is made subsequently in FIG. 5. In this design, the two chambers may be separated by a special seal.

A dual chamber tea bag made from a special paper or fabric can be used to eliminate the loss of THC and CBD properties in water. One chamber may contain professionally-produced tea with THC and CBD carrier, while the other chamber can be filled, by a dispensary, with freshly-dried marijuana.

To use, individuals can choose to break the seal between the two chambers or leave it intact, causing the two materials to be simultaneously infused and dispensed in boiling water. Dispensaries may customize the materials used in this device to satisfy the needs dictated by their patients' specific conditions and/or diseases. Such containers can help to partially produce products in FDA or Health Department approved labs and manufacturers, containing different necessary properties and formulations to improve the delivery of the cannabinoids. The containers can then be sent to dispensaries that may fill out the remaining portions of the containers with fluids or products freshly and specifically made for each client's needs, changing THC and CBD concentrations. Dispensaries may be required to include labels detailing the purity of the marijuana, concentration of cannabinoids, and pesticides used (or not used.)

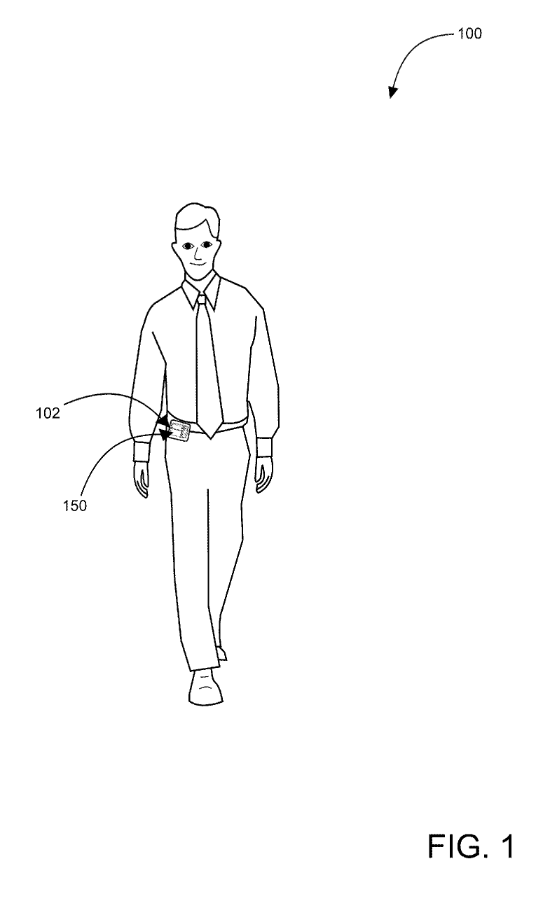

Referring to the drawings by numerals of reference there is shown in FIG. 1, a perspective view illustrating dual-chamber packaging system 100 for cannabis-infused products (medical marijuana-infused products 150 and/or cannabinoid fluids 152) in a stored/transported condition (by a user) according to an embodiment of the present invention.

A packaging apparatus 102 for medical marijuana-infused products 150 and edibles is shown comprising: a housing 104; at least one cap 106; at least a first chamber 110; at least a second chamber 120; and at least one nozzle 130. Cap 106 (or other suitable covering means) and housing 104 when coupled define an inner volume 105 of packaging apparatus 102; wherein first and second chambers (110 and 120, respectively) are effectively enclosed within inner volume 105 of housing 104 and cap(s) 106. Cap 106 when installed covers nozzles 130 to prevent damage to nozzles 130.

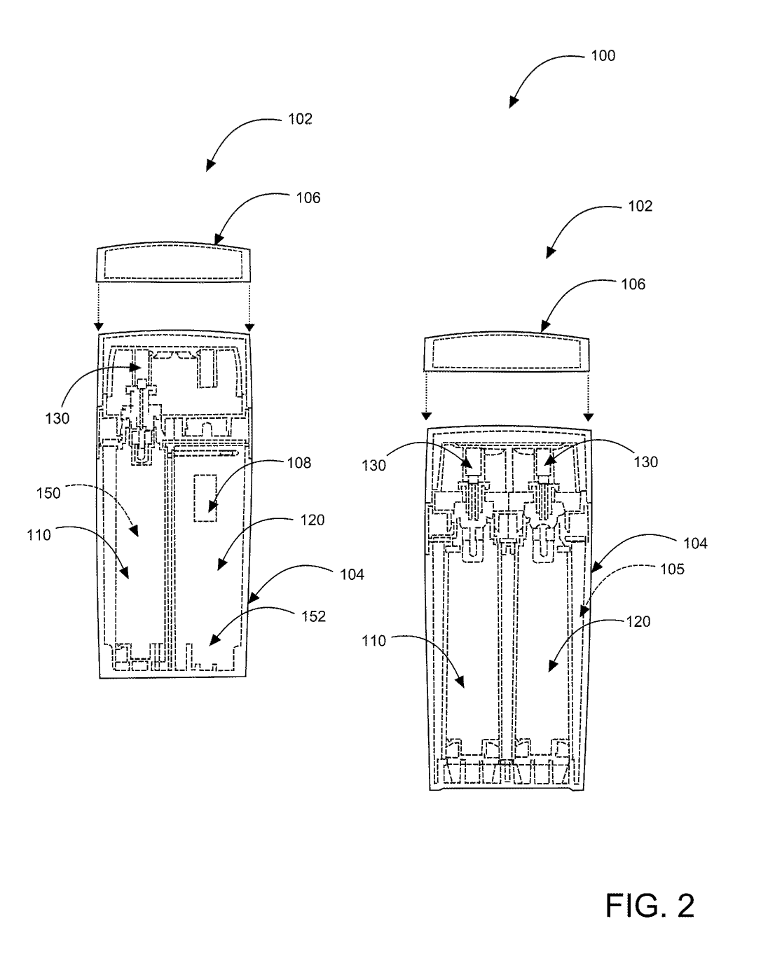

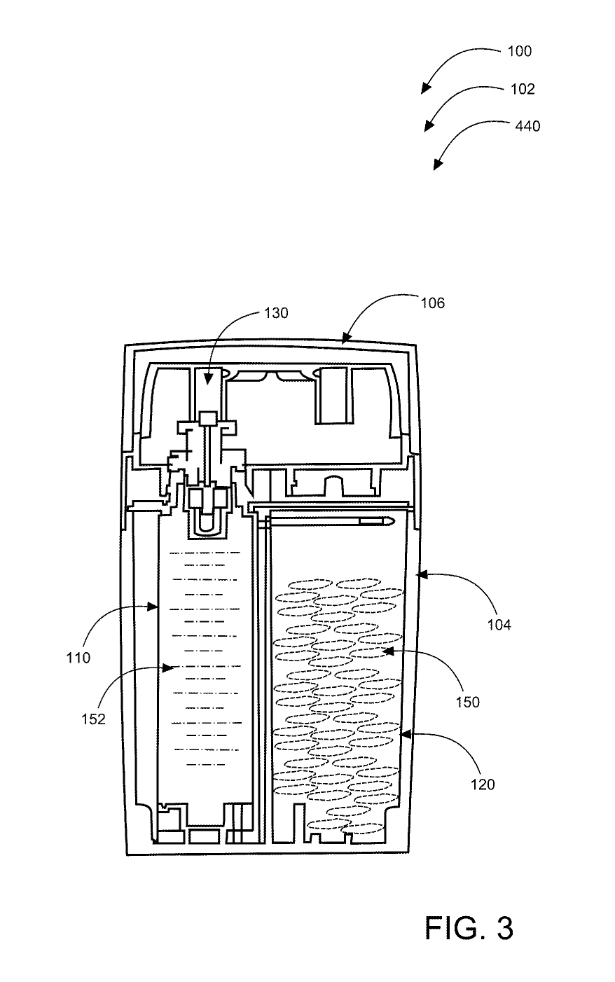

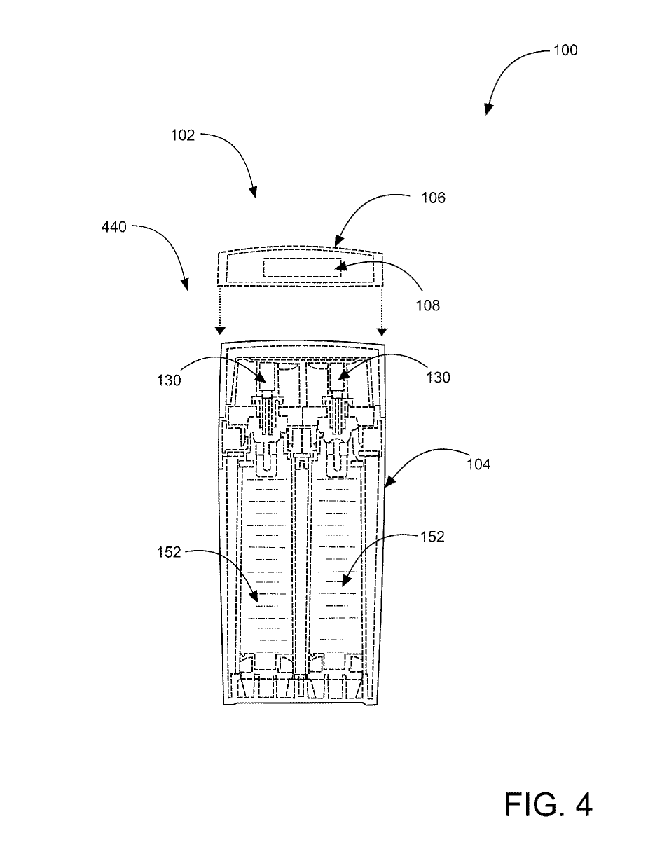

Referring now (in conjunction with FIG. 1) to FIGS. 2-4, starting with FIG. 2, a perspective view illustrating embodiments of dual-chamber packaging system 100 for cannabis-infused products and FIG. 3, a perspective view illustrating dual-chamber packaging system 100 for cannabis-infused products as used to sealably-contain cannabinoid fluid(s) 152 separately in a chamber from dry or gel products stored in another chamber (first chamber 110 and second chamber 120) according to an embodiment of the present invention of FIG. 2. FIG. 4, shows a perspective view illustrating dual-chamber packaging system 100 for cannabis-infused products (medical marijuana-infused products 150) as used to sealably-contain cannabinoid fluids 152 in each of the chambers (first chamber 110 and second chamber 120) according to an embodiment of the present invention of FIG. 2.

Using packaging apparatus 102 of dual-chamber packaging system 100, as partially described above, lotions and/or oils with THC and CBD enhancers and carriers comprising cannabinoids can be stored in first chamber 110; and cannabinoid fluid 152 may be contained in second chamber 120. Nozzles 130 allow a user to controllably dispense the lotion(s) and/or oils from first chamber 110 and cannabinoid fluid 152 (also comprising cannabinoids) from second chamber 120 simultaneously. Packaging apparatus 102 for medical marijuana-infused products 150 and edibles provides a storage and dispenser whereby a user can safely ingest or topically apply medical marijuana-infused products 150 and edibles to provide pain relief. Upon reading this specification, it should be appreciated that, under appropriate circumstances, considering such issues as user preferences, design preference, structural requirements, marketing preferences, cost, available materials, technological advances, etc., other cannabinoid products and dispensing means such as, for example, those with multiple chambers, other medical marijuana-infused products used, etc., may be sufficient.

Packaging apparatus 102 preferably comprises high-density polyethylene in preferred embodiments. Upon reading this specification, it should be appreciated that, under appropriate circumstances, considering such issues as user preferences, design preference, structural requirements, marketing preferences, cost, available materials, technological advances, etc., other materials such as, for example, other plastics, rubbers, and other suitable materials, etc., may be sufficient.

Packaging apparatus 102 when comprising a single-nozzle design (as shown in FIG. 3) can provide oral-mucosal delivery of cannabinoids. Nozzle 130, within this particular embodiment is usable to dispense lotions and/or oils and cannabinoids in first chamber 110 and second chamber 120 simultaneously. Those with ordinary skill in the art will now appreciate that upon reading this specification and by their understanding the art of cannabinoids as described herein, methods of using and dosages of medical marijuana-infused products to promote pain relief will be understood by those knowledgeable in such art.

In certain embodiments of packaging apparatus 102 first chamber 110 and second chamber 120 are separated by a breakable seal. The action of breaking the breakable seal causes the lotions and/or oils and cannabinoids to be simultaneously infused and dispensed in boiling water (or other.) Both first chamber 110 and second chamber 120 are refillable, such that the present invention is cost-effective in use, however it is advisable that first chamber 110 and second chamber 120 are thoroughly cleaned between uses. First chamber 110 and second chamber 120 are separate from each other within preferred embodiments shown; however they may form a single piece construction in alternate versions. First chamber 110 and second chamber 120 are suitable for sealingly-containing at least one liquid and/or solid.

Packaging apparatus 102 may comprise a dual chamber tea bag made from a special paper or fabric that can effectively be used to eliminate the loss of THC and CBD properties in water, thereby preserving its medicinal quality. In this particular embodiment first chamber 110 contains professionally-produced tea with a THC and CBD carrier, while second chamber 120 can be filled, by dispensary, with freshly-dried marijuana. In this way the present invention can be used to contain medical marijuana-infused products 150 in various states (liquids, solids and gases—if desired.) First chamber 110 and second chamber 120 may comprise color-coding and label(s) for ease of tracking and identifying user/use of medical marijuana-infused products 150.

Packaging apparatus 102 for medical marijuana-infused products 150 and edibles may permit an action of pumping two nozzles 130 separately allowing transdermal delivery of cannabinoids to the user. In a similar manner an action of pumping two nozzles 130 separately allows oral delivery of cannabinoids to the user. In these ways the present invention is multi-functional as a dispenser.

A tea bag, as referred to herein preferably comprises a regular (standard) tea bag with 2 chambers, with the exception that one side contains FDA approved carrier and tea, while the other contains dry cannabis. For oral application toothpaste, cannabinoid carrier may be held in one chamber and the other chamber with cannnabinoid fluid, since gums provide an excellent means for introducing cannabinoid into the patient-user in toothpaste form.

Kit 440 is disclosed herein including packaging apparatus 102 having housing 104; cap 106; at least one nozzle 130; first chamber 110; second chamber 120; labels 108 (labeling means); and a set of user instructions. Kit 440 may also include various medical marijuana-infused products 150 in a pre-package dispensible form. Dual-chamber packaging system 100 may be manufactured and provided for sale in a wide variety of sizes and shapes for a wide assortment of applications. Upon reading this specification, it should be appreciated that, under appropriate circumstances, considering such issues as design preference, user preferences, marketing preferences, cost, structural requirements, available materials, technological advances, etc., other kit contents or arrangements such as, for example, including more or less components (optionally multiple chambers, nozzles, etc.), customized parts, different labeling means, sealed or not sealed, and parts may be sold separately, etc., may be sufficient.

Referring now to FIG. 5, a flowchart 550 illustrating a method of use 500 for dual-chamber packaging system 100 according to an embodiment of the present invention of FIGS. 1-4.

A method of using (at least herein enabling method of use 500) a packaging apparatus 102 for medical marijuana-infused products 150 and edibles to ingest or topically apply medical marijuana to provide pain relief preferably comprises the steps of: step one 501 dispensing, ingesting and/or topically applying medical marijuana (medical marijuana-infused products 150) to provide pain relief via manipulating nozzles 130 (in the manner(s) as described above); and step two 502 refilling first chamber 110; and/or second chamber 120 of packaging apparatus 102 when medical marijuana-infused products 150 have been depleted. Refilling step may include the refilling of solids and/or liquids or combinations thereof as also described above. It should be noted that solids and various liquids may be used interchangeably within the figures shown.

It should be noted that the steps described in the method of use can be carried out in many different orders according to user preference. Upon reading this specification, it should be appreciated that, under appropriate circumstances, considering such issues as design preference, user preferences, marketing preferences, cost, structural requirements, available materials, technological advances, etc., other methods of use arrangements such as, for example, different orders within above-mentioned list, elimination or addition of certain steps, including or excluding certain maintenance steps, etc., may be sufficient.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A packaging apparatus for medical marijuana-infused products and edibles comprising:
a housing;
at least one cap;
at least a first chamber;
at least a second chamber;
at least one nozzle;
wherein said at least one cap and said housing when coupled define an inner volume of said packaging apparatus;
wherein said first and said second chambers are enclosed within said inner volume of said housing and said at least one cap;
wherein said at least one cap when installed covers said nozzles;
wherein lotions and/or oils with THC and CBD enhancers and carriers comprising cannabinoids can be stored in said first chamber;
wherein cannabinoid fluid may be contained in said second chamber;
wherein said nozzles allow a user to dispense said lotions and/or oils from said first chamber and said cannabinoid fluid also comprising said cannabinoids from said second chamber simultaneously or separately; and
wherein said packaging apparatus for medical marijuana-infused products and edibles provides a storage and dispenser whereby a user can safely ingest or topically apply said medical marijuana-infused products and edibles to provide pain relief.

2. The packaging apparatus for medical marijuana-infused products and edibles of claim 1 wherein said packaging apparatus comprises high-density polyethylene.

3. The packaging apparatus for medical marijuana-infused products and edibles of claim 1 wherein said packaging apparatus when comprising a single-nozzle design can provide oral-mucosal delivery of said cannabinoids.

4. The packaging apparatus for medical marijuana-infused products and edibles of claim 1 wherein a single said nozzle is usable to dispense said lotions and/or oils and said cannabinoids in said first chamber and said second chamber simultaneously.

5. The packaging apparatus for medical marijuana-infused products and edibles of claim 1 wherein said first chamber and said second chamber are separated by a breakable seal.

6. The packaging apparatus for medical marijuana-infused products and edibles of claim 1 wherein said first chamber and said second chamber are refillable.

7. The packaging apparatus for medical marijuana-infused products and edibles of claim 1 wherein said first chamber and said second chamber are separate from each other.

8. The packaging apparatus for medical marijuana-infused products and edibles of claim 1 wherein a dual chamber tea bag made from a special paper or fabric can be used to eliminate the loss of THC and CBD properties in water.

9. The packaging apparatus for medical marijuana-infused products and edibles of claim 1 wherein said first chamber contains professionally-produced tea with a THC and CBD carrier, while said second chamber can be filled, by dispensary, with freshly-dried marijuana.

10. The packaging apparatus for medical marijuana-infused products and edibles of claim 5 wherein action of breaking said breakable seal causes said lotions and/or oils and said cannabinoids to be simultaneously infused and dispensed in boiling water.

11. The packaging apparatus for medical marijuana-infused products and edibles of claim 1 wherein said first chamber and said second chamber are suitable for sealingly-containing at least one liquid.

12. The packaging apparatus for medical marijuana-infused products and edibles of claim 1 wherein said first chamber and said second chamber are suitable for sealingly-containing at least one solid.

13. The packaging apparatus for medical marijuana-infused products and edibles of claim 1 wherein said first chamber and said second chamber are suitable for sealingly-containing at least one liquid and/or solid.

14. The packaging apparatus for medical marijuana-infused products and edibles of claim 1 wherein said first chamber and said second chamber comprise color-coding.

15. The packaging apparatus for medical marijuana-infused products and edibles of claim 1 wherein said first chamber and said second chamber comprise labels.

16. The packaging apparatus for medical marijuana-infused products and edibles of claim 1 wherein an action of pumping said two nozzles separately allows transdermal delivery of said cannabinoids to the user.

17. The packaging apparatus for medical marijuana-infused products and edibles of claim 1 wherein an action of pumping said two nozzles separately allows oral delivery of said cannabinoids to the user.

18. A packaging apparatus for medical marijuana-infused products and edibles comprising:
a housing;
a cap;
a first chamber;
a second chamber;
at least one nozzle, said nozzle(s) able to dispense atomized liquid and/or non-atomized fluid;
wherein said cap and said housing when coupled define an inner volume of said packaging apparatus;
wherein said first and said second chambers are enclosed within said inner volume of said housing;
wherein said cap when installed covers said nozzle(s);
wherein lotions and/or oils with THC and CBD enhancers and carriers can be stored in said first chamber;
wherein cannabinoid fluid may be contained in said second chamber;
wherein said packaging apparatus comprises high-density polyethylene;
wherein said first chamber and said second chamber are separated by a breakable seal;
wherein said first chamber and said second chamber are refillable;
wherein said first chamber and said second chamber are separate from each other;
wherein said first chamber and said second chamber are suitable for sealingly-containing at least one liquid and/or solid;
wherein said first chamber and said second chamber comprise color-coding;
wherein said first chamber and said second chamber comprise labels; and
wherein said packaging apparatus for medical marijuana-infused products and edibles provides a means for a user to safely ingest and/or topically apply said medical marijuana-infused products and edibles.

19. A packaging apparatus for medical marijuana-infused products and edibles of claim 18 comprising a kit including said packaging apparatus having said housing; said cap; said at least one nozzle; said first chamber; said second chamber; said labels; and a set of user instructions.

20. A method of using a packaging apparatus for medical marijuana-infused products and edibles to ingest or topically apply medical marijuana to provide pain relief comprising the steps of:
dispensing, ingesting and/or topically applying said medical marijuana to provide pain relief via manipulating nozzles; and
refilling a first chamber; and a second chamber of said packaging apparatus when said medical marijuana-infused products have been depleted.
